(19) United States
(12) Patent Application Publication
(10) Pub. No.: US 2023/0080136 A1
(43) Pub. Date: Mar. 16, 2023

(54) IMPLANTABLE URINARY CONTINENCE DEVICE WITH HELICAL ANCHOR

(71) Applicant: Uromedica, Inc., Plymouth, MN (US)

(72) Inventors: John H. Burton, Minnetonka, MN (US); Timothy C. Cook, Wayzata, MN (US)

(21) Appl. No.: 17/931,730

(22) Filed: Sep. 13, 2022

Related U.S. Application Data
(60) Provisional application No. 63/261,171, filed on Sep. 14, 2021.

(51) Int. Cl. A61F 2/00

(52) U.S. Cl. CPC A61F 2/0013; A61F 2/004; A61F 2/0045; A61B 2017/00004

(57) ABSTRACT

An implantable device includes a conduit, an adjustable membrane element coupled to the conduit near the front end of the conduit for controllable coaptation of a body lumen, such as coaptation of a urethra as treatment for urinary incontinence, and a helix coupled to the front end of the conduit. The helix functions as a fixation mechanism to anchor the implantable device to the tissue. The implantable device can be inserted into tissue using a sheath and can be rotated with the sheath by partially inflating the adjustable membrane element placed in a front end portion of the sheath, and the helix can be turned into the tissue by rotating the sheath.

### CLAIM OF PRIORITY

This application claims the benefit of priority under 35 U.S.C. § 119(e) of U.S. Provisional Patent Application Ser. No. 63/261,171, entitled “IMPLANTABLE URINARY CONTINENCE DEVICE WITH HELICAL ANCHOR”, filed on Sep. 14, 2021, which is herein incorporated by reference in its entirety.

### TECHNICAL FIELD

This document relates generally to implantable medical devices and more particularly to a device with a helix for anchoring a device for treating urinary incontinence to tissue after its implantation in a patient.

### BACKGROUND

An example of an implantable device for treating urinary incontinence includes an adjustable membrane element, such as a balloon, connected to a rear port with a conduit. The implantable device can be implanted in a patient with the adjustable membrane element placed adjacent to the patient's urethra and the rear port placed underneath the patient's skin by minimally invasive surgery. The adjustable membrane element can be adjusted during and after the surgery by injecting fluid into the rear port or extracting fluid from the rear port percutaneously using a needle. In an exemplary treatment, two of such implantable devices are placed in the patient such that the two adjustable membrane elements provide pressure and support at the patient's bladder neck to protect against accidental leaking of urine in cases such as stress urinary incontinence (e.g., leaking during sneeze, cough, or physical activity). The efficacy of this treatment depends on accurate placement of the adjustable membrane element at a target site in the patient, adjustment of the adjustable membrane element after the placement, and maintaining the position of the adjustable membrane element over time.

### SUMMARY

An implantable device includes a conduit, an adjustable membrane element coupled to the conduit near the front end of the conduit for controllable coaptation of a body lumen, such as coaptation of a urethra as treatment for urinary incontinence, and a helix coupled to the front end of the conduit. The helix functions as a fixation mechanism to anchor the implantable device to the tissue. The implantable device can be inserted into tissue using a sheath and can be rotated with the sheath by partially inflating the adjustable membrane element placed in a front end portion of the sheath, and the helix can be turned into the tissue by rotating the sheath.

This summary is an overview of some of the teachings of the present application and not intended to be an exclusive or exhaustive treatment of the present subject matter. Further details about the present subject matter are found in the detailed description and appended claims. The scope of the present invention is defined by the appended claims and their legal equivalents.

### BRIEF DESCRIPTION OF THE DRAWINGS

FIG. 1 is a perspective view of an implantable device and a syringe source for providing a flowable material to an adjustable membrane element of the implantable device, according to an embodiment of the present subject matter.

FIG. 2 is a longitudinal cross-sectional view of the implantable device shown in FIG. 1, according to an embodiment of the present subject matter.

FIG. 3 is a cross-sectional view taken along line 3-3 of FIG. 2, according to an embodiment of the present subject matter.

FIG. 4 shows the implanted device of FIG. 1 after being placed at a desired location in a patient and expanded to displace body tissue toward a body lumen for causing adjustable restriction of the body lumen, according to an embodiment of the present subject matter.

FIG. 5 is a longitudinal cross-sectional view of another implantable device, according to an embodiment of the present subject matter.

FIG. 6 is a cross-sectional view taken along line 6-6 of FIG. 5, according to an embodiment of the present subject matter.

FIG. 7 illustrates a sheath used for implantation of an implantable device, according to an embodiment of the present subject matter.

FIG. 8 illustrates a trocar used with the sheath of FIG. 7, according to an embodiment of the present subject matter.

FIG. 9 illustrates an assembly the sheath of FIG. 7 and the trocar of FIG. 8, according to an embodiment of the present subject matter.

FIG. 10 is an illustration, including a broken-out view, showing introduction of an implantable device into tissue of a patient using the assembly of the sheath of FIG. 7 and the trocar of FIG. 8, to provide for coaptation of a urethra, according to an embodiment of present subject matter.

FIG. 11 is an illustration, including a broken-out view, showing a pair of implantable devices placed in the patient to provide for coaptation of the urethra, according to an embodiment of present subject matter.

FIG. 12 an illustration, including a broken-out view, showing adjustment of the coaptation of the urethra after the pair of implantable devices are placed in the patient as shown in FIG. 11, according to an embodiment of present subject matter.

FIG. 13 is an illustration of an implantable device, a push wire, and a sheath, according to an embodiment of present subject matter.

FIG. 14 is an illustration of another implantable device, the push wire, and the sheath, according to an embodiment of present subject matter.

FIGS. 15-19 are illustrations of a method for placing an implantable device into a patient using a sheath, according to an embodiment of present subject matter.

FIG. 15 shows the implantable device being partially placed in the sheath.

FIG. 16 shows an adjustable membrane element of the implantable device being advanced into a front portion of the sheath and partially inflated to allow turning a helix of the implantable device into tissue.

FIG. 17 shows the sheath being partially withdrawn to allow inflation of the adjustable membrane element.

FIG. 18 shows the adjustable membrane element being inflated for coaptation of a body lumen of the patient.

FIG. 19 shows the sheath being withdrawn to allow the sheath to be separated from the implantable device.

FIGS. 20-21 are illustrations of a scenario in performing the method of FIGS. 15-19 and device features related to the scenario, according to an embodiment of present subject matter.

FIG. 20 shows the adjustable membrane element of the implantable device protruding from a slot of the sheath.

FIG. 21 is a longitudinal cross-sectional view of the adjustable membrane element of the implantable device protruding from the slot of the sheath.

### DETAILED DESCRIPTION

The following detailed description of the present subject matter refers to subject matter in the accompanying drawings which show, by way of illustration, specific aspects and embodiments in which the present subject matter may be practiced. These embodiments are described in sufficient detail to enable those skilled in the art to practice the present subject matter. References to “an”, “one”, or “various” embodiments in this disclosure are not necessarily to the same embodiment, and such references contemplate more than one embodiment. The following detailed description is demonstrative and not to be taken in a limiting sense. The scope of the present subject matter is defined by the appended claims, along with the full scope of legal equivalents to which such claims are entitled.

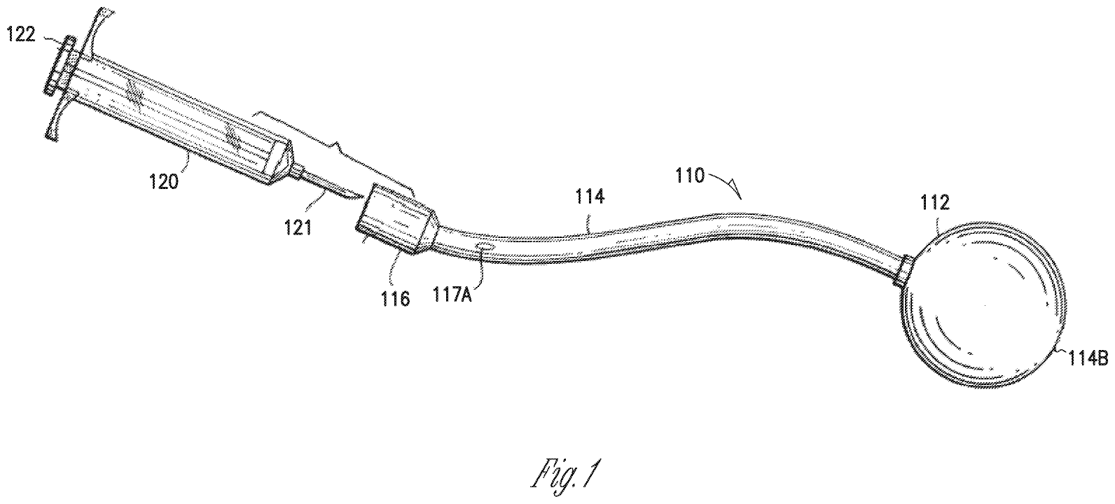

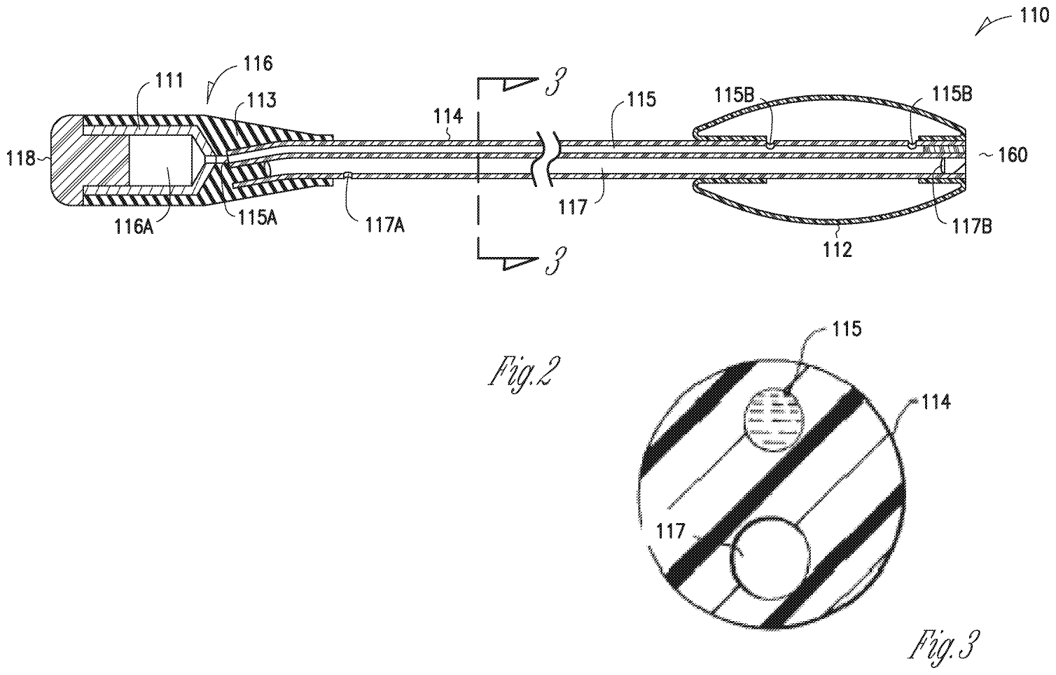

This document discusses, among other things, a mechanism and tool for fixation of an implantable device to surrounding tissue for treating urinary incontinence. The implantable device can include, for example, an adjustable membrane element connected to a rear port with a conduit that has a lumen providing for fluid communication between a chamber of the adjustable membrane element and an interior cavity of the rear port. Various structural elements of the implantable device (e.g., an implantable device 110 shown in FIG. 1) discussed in this document may each be referred to by various terms. The “adjustable membrane element” (e.g., the adjustable membrane element 112 shown in FIG. 1) can also be referred to as, for example, an adjustable element, an expandable element, an expandable membrane element, a forward expandable membrane element, a balloon, or an adjustable balloon. The “conduit” the conduit 114 shown in FIG. 1) can also be referred to as, for example, a central conduit element, a device conduit, a connecting conduit, a connecting conduit tube, or a tubular elongate body. The “rear port” (e.g., the rear port 116 shown in FIG. 1) can also be referred to as, for example, a rearward port portion or a rear port element. The “lumen” (e.g., the first lumen 115 and the second lumen 117 shown in FIG. 2) can also be referred to as, for example, a passageway, an inner passageway, or an interior passageway.

In an example, the implantable device includes an adjustable balloon connected to a port with a conduit. The balloon is placed adjacent the urethra to exert non-circumferential compression upon the urethral wall. The effectiveness of the therapy depends on proper positioning of the balloon at a target site in a patient's body, such as in the retropubic space near the urethra-vesical junction above the urogenital diaphragm in close proximity to the urethral walls. When two balloons (e.g., of two implantable devices) are used, their preferred positioning is usually symmetrical and lateral with respect to the urethra. Medical imaging techniques such as fluoroscopy or transrectal ultrasonography (TRUS) can be used to aid the positioning of the balloon(s). Sensors incorporated into the implantable device(s) and/or one or more surgical tools can also be used to aid the positioning of the balloon(s), such as discussed in U.S. patent application Ser. No. 16/450,246, filed on Jun. 24, 2019, assigned to UroMedica, Inc., which is incorporated by reference herein in its entirety.

During the implantation procedure, the implantable device(s) is(are) placed in the patient with the balloon(s) positioned and fixed in place at the target site(s). The balloon(s) is(are) only slightly inflated, typically between 0.5 and 1.5 cc, for a period of 4 to 6 weeks to allow tissue encapsulation in order to stabilize the balloon(s) at its(their) target site(s). In particular, without encapsulation the implantable device(s) is(are) prone to migrate down the dilation path through which the implantable device(s) was(were) implanted. For optimal effect it is important that the balloon(s) be maintained above the pelvic floor. Thus, it is very important that fixation occur during this implantation procedure. After the encapsulation, the patient will go through one or more adjustment procedures during which the volume of fluid in the balloon(s) is adjusted to obtain and maintain urinary continence without causing undesirable obstruction.

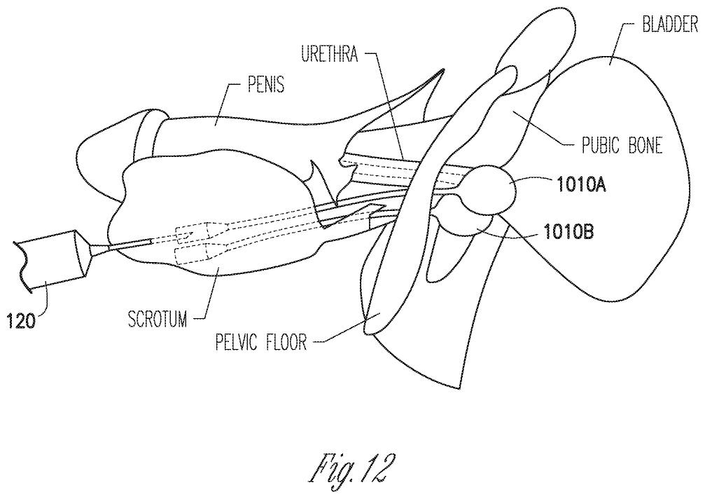

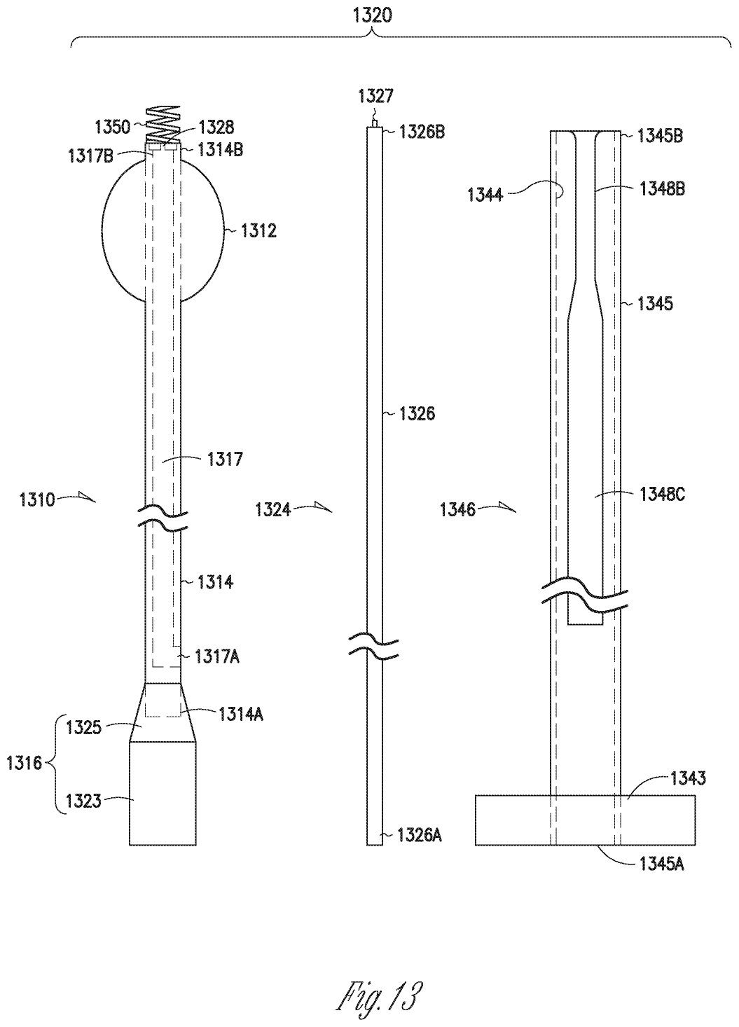

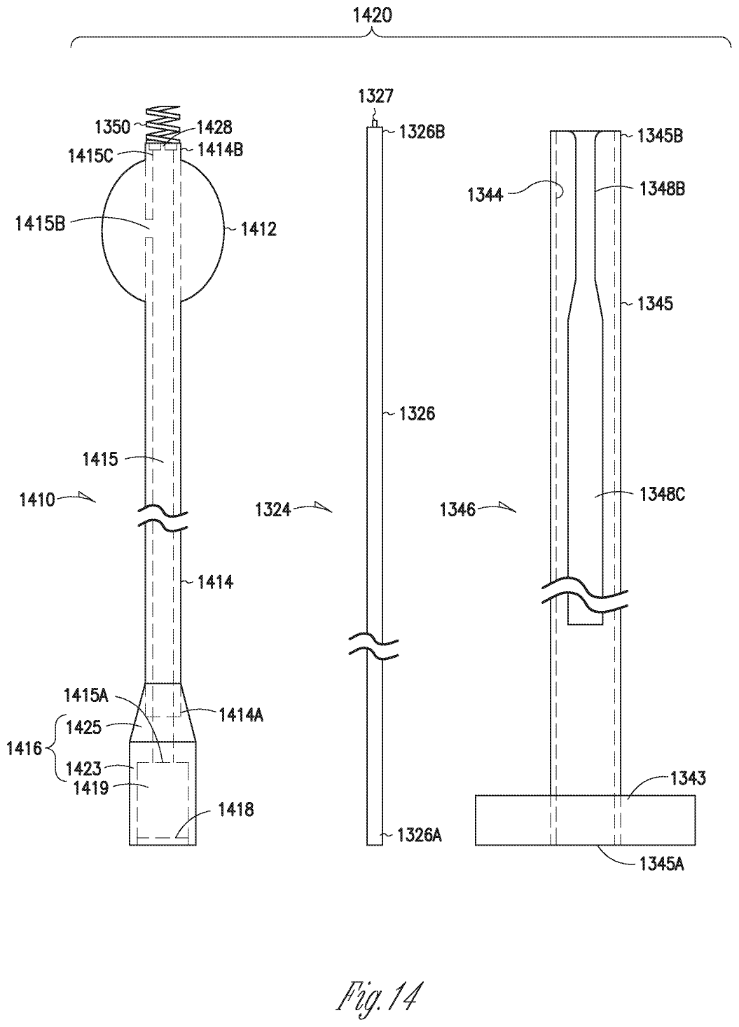

The present subject matter provides an implantable device for treating urinary incontinence that has a helical fixation mechanism for preventing a balloon of the implantable device from unwanted displacement. FIGS. 1-9 illustrate various embodiments of an implantable device into which the helical fixation mechanism can be incorporated and surgical tools used for placing the implantable device into a patient. The various embodiments of the implantable device and the surgical tools are illustrated in FIGS. 1-9 and discussed below by way of example, and not by way of restriction. These examples as well as additional examples of the implantable device and the surgical tool are discussed in U.S. Pat. Nos. 5,964,806, 6,045,498, 6,419,624, 6,579,224, 8,926,494, and 9,861,384, all assigned to UroMedica, Inc., which are incorporated by reference herein in their entireties. FIGS. 10-12 illustrate an example of placement and adjustment of a pair of the implantable devices for urethral coaptation in a post-prostatectomy patient. FIGS. 13-19 illustrate the helical fixation mechanism incorporated onto the implantable device and a process for placing the implantable device into tissue of a patient and anchoring the implantable medical device to the tissue.

FIG. 1 is a perspective view of an implantable device 110 and a syringe 120, according to an embodiment of the present subject matter. FIG. 2 is a longitudinal cross-sectional view of the implantable device 110 FIG. 3 is a cross-sectional view of the implantable device 110 taken along line 3-3 of FIG. 2. The implantable device 110 includes an adjustable membrane element (also referred to as a balloon) 112, shown in its expanded state in FIG. 1, which is attached pressure-tightly to an elongate conduit 114. The conduit 114 has a front end 160. In one embodiment, the peripheral surface of the conduit 114 is connected to and sealed to the adjustable membrane element 112. In one embodiment, the adjustable membrane element 112 includes a continuous wall having an inner surface defining a chamber.

The conduit 114 includes a first lumen 115 and a second lumen 117 (as shown in FIGS. 2 and 3). In one embodiment, the first lumen 115 extends longitudinally in the conduit 114 from a first opening 115A. to one or more second openings 115B (e.g., two openings as shown in FIG. 2). The second opening(s) 115B is(are) in fluid communication with the chamber of adjustable membrane element 112 for adjustably expanding or contracting the adjustable membrane element 112 by flowable material introduced through the first opening 115A. To prevent leakage of the fluid from the adjustable membrane element 112, the first lumen 115 has a closed end at or near the front end 160 of the conduit 114. The closed end can be formed by sealing the front end of the first lumen 115, for example, using silicone adhesive. Alternatively, the first lumen 115 can be closed by manufacturing it to end before reaching the front end of the conduit 1014.

The second lumen 117 extends longitudinally along the conduit 114 from an inlet 117A to a closed end 117B at the front end 160. In one embodiment, the second lumen 117 and the inlet 117A are each of sufficient diameter to receive a push wire (also referred to as a push rod) that can be used to advance the implantable device 110 in the tissue.

The implantable device 110 further includes a rear port 116, which is coupled to the rear end of the conduit 114. In one embodiment, this includes a cavity 116A and an elastic septum 118. The cavity 116A is coupled to and in fluid communication with the first lumen 115 at the first opening 115A. The elastic septum 118 allows for access to the cavity 116A using a needle (such as the needle 121 shown in FIG. 1) for introducing and/or withdrawing fluid to expand (inflate) and/or contract (deflate) the adjustable membrane element 112. The diameter of the elastic septum 118 can be slightly larger than the diameter of the cavity 116A to produce compression to the elastic septum 1018 for better sealing. A syringe 120 including a hollow needle 121 and a rear axially-movable plunger 122 is provided for injecting a suitable flowable material into or drawing the suitable flowable material from the implantable device 110 through the rear port 116 to expand or contract, respectively, the adjustable membrane element 112. In various embodiments, the flowable material can be, for example, normal saline, polymer gels such as silicone gels or hydrogels of polyvinylpyrrolidone, polyethylene glycol, or carboxy methyl cellulose, or high viscosity liquids such as hyaluronic acid, dextran, polyacrylic acid, or polyvinyl alcohol. When desired, the flowable material can be made radiopaque (such as isotonic contrast media) so that the degree of membrane inflation can be viewed by x-ray or be echogenic so that it can be viewed by ultrasound.

In one embodiment, as illustrated in FIG. 2, the rear port 116 includes a titanium port liner 111 and an overmold 113. The port liner 111 surrounds the cavity 116A and a portion of the septum 118 to prevent the needle 121 from piercing through the rear port 116 from the inside or in case needle 121 has been misdirected from the outside. The inner diameter of the port liner 111 can be slightly smaller than the enclosed portion of the septum 118 to provide compression for better sealing. As shown in FIG. 2, the port liner 111 includes a cylindrical portion that surrounds the cavity 116A and a cap connected to the cylindrical portion. The cap has a hole that allows for the fluid communication between the cavity 116A and the first lumen. The hole can have a smaller diameter than that of the needle to prevent the needle from forward penetration beyond the cavity 116A, The overmold 113 can be made of a silicone or biostable segmented polyurethane elastomer and molded over the port liner 111 and a real portion of the conduit 114 to connect the rear port 116 to the conduit 114. The overmold 113 includes a tapered portion that functions as a strain relief, which protects the conduit 114 including the connection between the conduit 114 and the rear port 116 from, for example, damage that may result from cyclic bending due to body movements while the implantable device 110 is implanted in the patient or breakage that may result from puling during removal of the implantable device 110 from tissue.

The entire implantable device 110, including the adjustable membrane element 112 is formed of biocompatible materials including, for example, a silicone or polyurethane-based elastomer and metals such as titanium or tantalum, suitable for long-term implant. Optionally, the conduit 114 and the rear port 116 can be formed as a unitary construction. Optionally, the implantable device 110 includes one or more elastic portions each constructed of biostable segmented polyurethane, which is polyurethane with flexible segments of macrodiols chosen for biostability. This biostability is the ability of a polymer to resist degradation such as by stress cracking in the body over time expected of a long-term implant. Macrodiols of silaxane, polyether, and polycarbonate are known to impart biostability to segmented polyurethanes and can be used in any combination to impart properties such as toughness, resistance to cuts, wear and lack of permeability superior to silicone. In addition, being thermoplastic, the macrodiols allow for blow molding the adjustable membrane element 112 at a significant reduction in cost and possibly use of additive manufacturing such as 3D printing. The adjustable membrane element 112 can be adhered to the conduit 114 using a suitable adhesive or by means such as sonic welding or solvent bonding. An example of a silicone-based material includes polydimethylsiloxane (PDMS), having various formulation depending on the intended application, such as for injection molding, extrusion, in a dispersion for dip molding on a mandril, or as an adhesive.

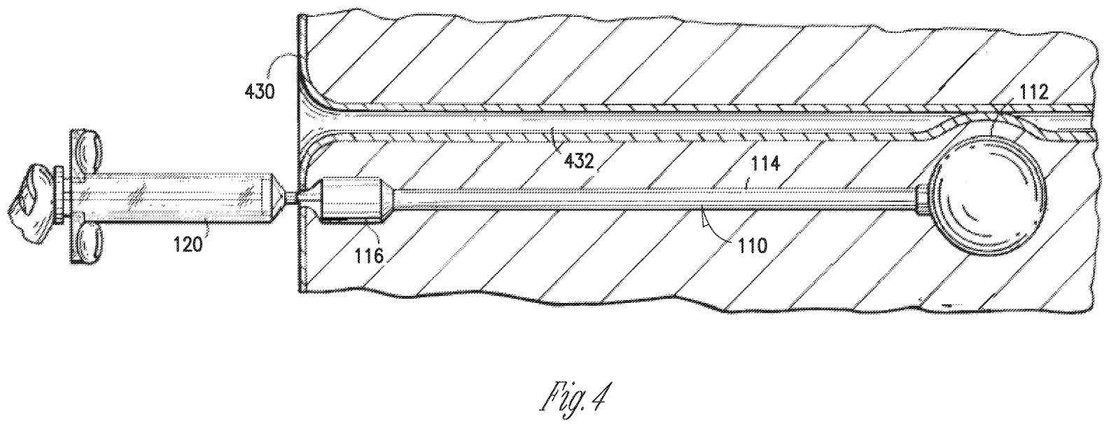

FIG. 4 shows the implanted device 110 after being placed at a desired location in a patient and expanded to displace body tissue toward a body lumen 432 for causing adjustable restriction of the body lumen 432, according to an embodiment of the present subject matter. After the implantable device 110 has been placed in the patient (e.g., using a method with surgical tools as discussed below with reference to FIGS. 7-10) such that the adjustable membrane element 112 in its contracted state is in the desired position adjacent to the body lumen 432, the body lumen 432 can be restricted to a desired degree by piercing septum 118 with the needle 121 of the syringe 120 and injecting the flowable material through the first lumen 115 into the adjustable membrane element 112. The physician can determine the desired degree of restriction of the body lumen 432 by means such as infusing fluid through the body lumen 432 past the restriction and measuring the back pressure. In one embodiment, the body lumen is the urethra, as further discussed below with reference to FIGS. 10-12.

After the implantable device 110 has been properly positioned with the adjustable membrane element 112 located near the body lumen 432 and the septum 118 in the rear port 116 located near the skin 430, the device is injected with the flowable material from the syringe 120. The adjustable membrane element 112 can be inflated to a certain extent and then deflated to an extent suitable for encapsulation of the adjustable membrane element 112 by body tissue.

The present subject matter provides the implantable device 110 with adjustability of the membrane expansion post-operatively. This adjustability is effected because the septum 118 is located remote from the adjustable membrane element 112 but near and under the patient's skin, for example in the scrotum of a male patient or labia of a female patient. The rear port 116 and septum 118 are located by, for instance, manual palpation of the skin region and the needle 121 of the syringe 120 is inserted through the skin and the septum 118 to add or remove the Plowable material from the adjustable membrane element 112, thus increasing or decreasing the restriction of the body lumen 432.

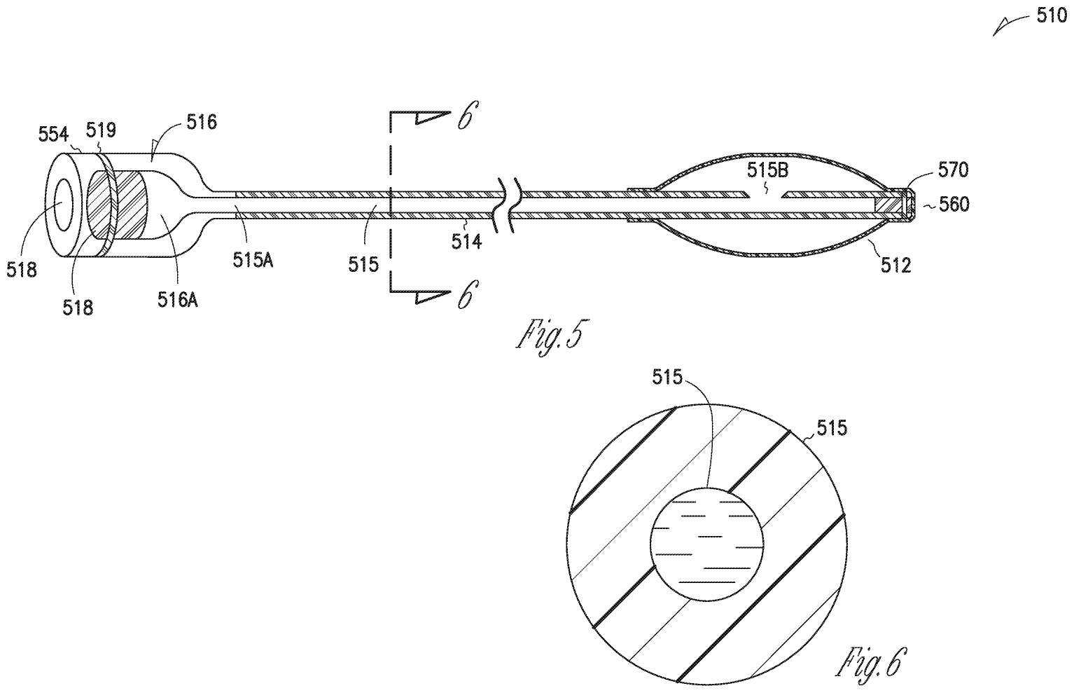

FIG. 5 is a longitudinal cross-sectional view of an implantable device 510, according to an embodiment of the present subject matter. FIG. 6 is a cross-sectional view of the implantable device 510 taken along line 6-6 of FIG. 5. The implantable device 510 includes an adjustable membrane element (also referred to as a balloon) 512 and an elongate conduit 514, where the conduit 514 includes at least a first lumen 515 which extends longitudinally in the conduit 514 from a first opening 515A at a rear end (also referred to as a proximal end) of the conduit to a second opening 515B.

The implantable device 510 further includes a rear port 516, where the rear port 516 is coupled to the rear end of the conduit 514. In one embodiment, the rear port 516 is coupled to the rear end of the elongate body 514 using chemical adhesives, or alternately, depending on the materials, using sonic welding, solvent bonding and/or other techniques as are known in the art. In an additional embodiment, the rear port 516 and the conduit 514 are formed together in a polymer molding process, such as liquid injection molding or overmolding.

The rear port 516 includes a cavity 516A, where the cavity 516A is in fluid communication with the first opening 515A of the conduit 514. In one embodiment, the rear port 516 also includes an elastic septum 518 through which the cavity 516A is accessed, where the elastic septum 518 is self-sealing after repeated pierces, for example, with a needle. In one embodiment, the elastic septum 518 is retained in the rear port 516 by a clamp ring 519 located around the rear port 516. In one embodiment, the clamp ring 519 is made of a biocompatible material, such as, for example, titanium. In one embodiment, the elastic septum 518 is made of a biocompatible material, such as, for example, silicone or biostable segmented polyurethane. The rear port 516 has an outer diameter defined by an outer surface 554 of the rear port 516. In one embodiment, the rear port 516 has an outer diameter between 2 and 15 millimeters, with 5.7 millimeters being a specific example.

Once the implantable device 510 is positioned within a body, the adjustable membrane element 512 is inflated by releasably connecting a flowable material source to the rear port 516. In one embodiment, the flowable material source includes a syringe with a non-coring needle, such as the syringe 120 with the needle 121, where the needle is inserted through the elastic septum 518. A measured supply of fluid volume can be introduced into the implantable device 510, and the adjustable membrane element 112 expands or contracts due to a volume of the flowable material introduced into the cavity 516A of the rear port 516 from the flowable material source. The adjustable membrane element 512 is then used to at least partially and adjustably restrict the body lumen. Once the adjustable membrane element 512 has been inflated, the needle is withdrawn from the septum 518 of the rear port 516.

in an additional embodiment, a detectable marker 570 is imbedded in the implantable device 510. For example, the detectable marker 570 is located at the front end (also referred to as a distal end) 560 of the conduit 514. In one embodiment the detectable marker 570 is located in a lumen of the conduit 514. The detectable marker 570 allows the front end 560, and thus the front end of the adjustable membrane element 512, to be located within the tissue of a patient using any number of visualization techniques which employ electromagnetic energy as a means of locating objects within the body. In one embodiment, the detectable marker 570 is constructed and located to allow for visualization and orientation of the adjustable membrane element 512 in the tissue of the patient. In one embodiment, the detectable marker 570 is constructed of radiopaque tantalum and can be visualized with X-ray. In one embodiment, the entire conduit 514 is made radiopaque by, for example, dispersing tantalum powder in the conduit 514. The implantable device 510 can formed of biocompatible materials in the same or substantially similar manner as discussed above for the implantable medical device 110. Likewise, a detectable marker can be placed in substantially the same way, and serve substantially the same purposes, as discussed above for the implantable medical device 110.

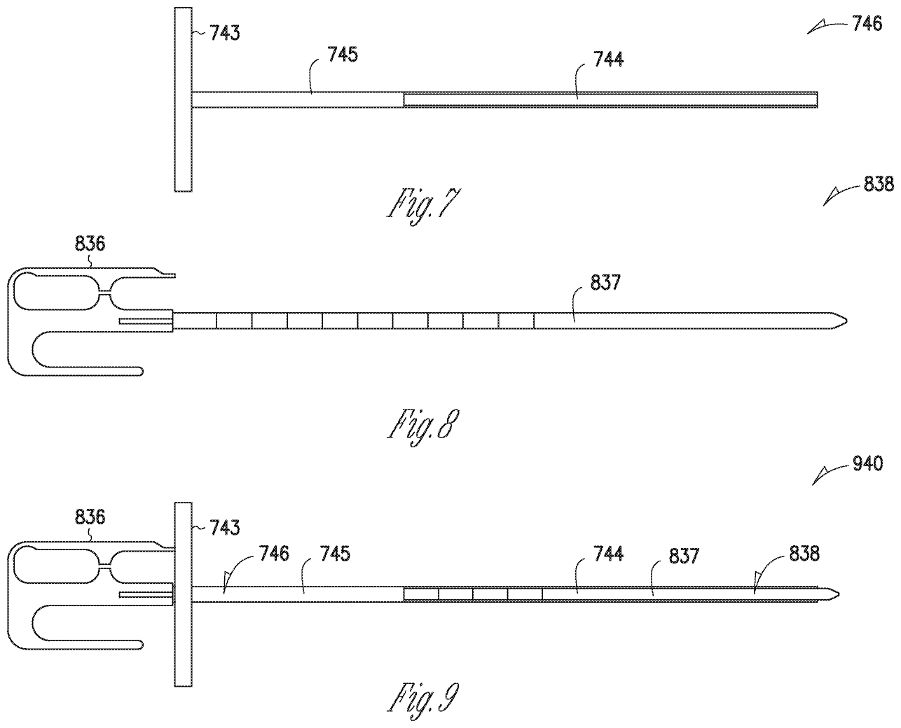

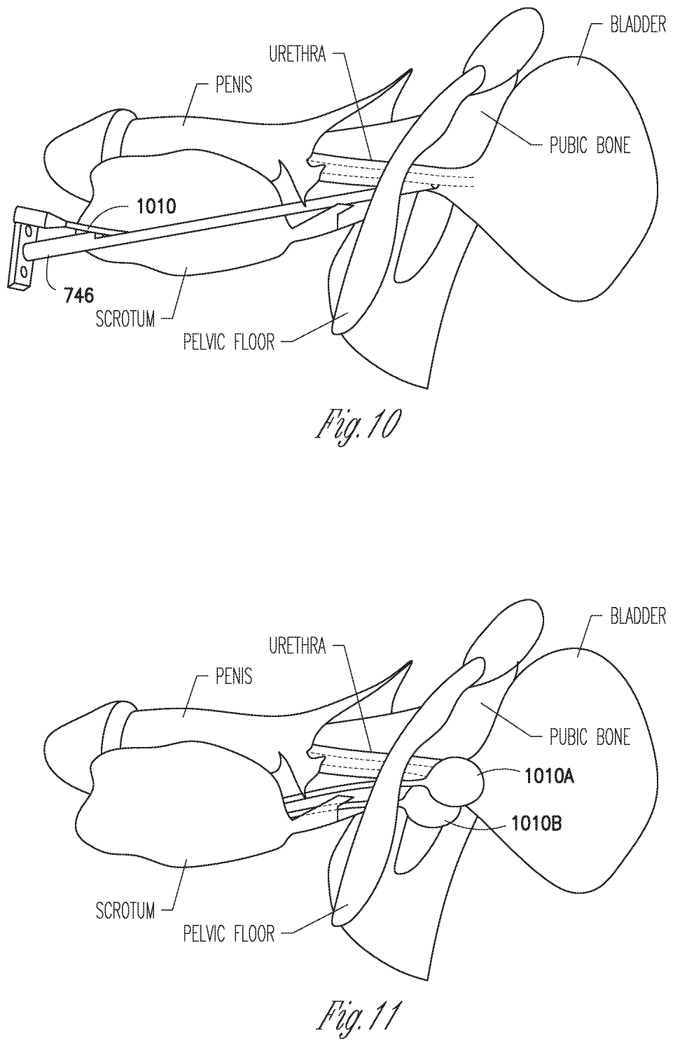

FIGS. 7-9 illustrate a surgical tool kit for placement of an implantable device, such as the implantable device 110 and 510, in tissue of the patient. The surgical tool kit includes a sheath 746 (shown in FIG. 7) and a trocar 838 (shown in FIG. 8). FIG. 9 shows an assembly 940 including the sheath 746 and the trocar 838.

The trocar 838 includes an elongate member (shaft) 837 and a handle portion 836. In one embodiment, the trocar 838 is disposable (i.e., not intended for reuse or not suitable for reuse, e.g., not suitable for cleansing and re-sterilization after use). In another embodiment, the trocar 838 is reusable (i.e., can be cleansed and re-sterilized after each use). The elongate member 837, in various embodiments, is sterilizable. In additional embodiments, the handle portion 836 is also sterilizable. In another embodiment, the trocar 838 includes steam sterilizable components. Various embodiments incorporate materials known to provide for such function, such as surgical grade stainless steel. Multiple embodiments are contemplated by the present subject matter. In each embodiment, one or more materials are used in constructing the elongate member 837. In each embodiment, one or more materials are used in constructing handle portion 836.

The trocar 838 has a proximal end and a distal end, with the handle portion located at the proximal end. In one embodiment, the trocar 838 has a sharp tip at the distal end. In another embodiment, the trocar 838 has a blunt tip at the distal end. In one embodiment, the trocar 838 having the sharp tip and the trocar 838 having the blunt tip are both provided for implanting the device such as implantable device 110 and 510.

The sheath 746 includes an elongate member (shaft) 745 and a handle portion 743. In one embodiment, the sheath 746 is disposable (i.e., not intended for reuse or not suitable for reuse, e.g., not suitable for cleansing and re-sterilization after use). This may provide for cost effectiveness when the sheath 746 is subjected to damage during use (e.g., to be modified to facilitate its separation from the implantable device during an implantation procedure). In another embodiment, the sheath 746 is reusable (e.g., can be cleansed and re-sterilized after each use). The elongate member 745 is trough (U- or C-) shaped across its diameter, in one embodiment. In various embodiments, the elongate member 745 includes tubing which has a slot opening running at least part of the way down its length. The elongate member 745 has a cross section which is curved in various embodiments. As such, these embodiments define a channel 744 of the sheath 746. In various embodiments, a removable trocar is sized for slidable disposition in the elongate member of the sheath 746, via an opening in the handle portion 743. In various embodiments, one or more materials are used in constructing the elongate member 745. In each embodiment, one or more materials are used in constructing handle portion 743. Examples of such one or more materials include stainless steel and various suitable polymers.

The trocar 838 is inserted into the sheath 746 to form the assembly 940, as shown in FIG. 9. In one embodiment, the trocar 838 having the sharp tip is used for penetrating tissue of the patient, such as when the physician grasps the handle 836 and maneuvers the trocar 838, distal portion first, through an incision, any scar tissue that may be present, the facia of the pelvic floor and toward an implant site located proximal to the bladder neck or prostate (if present) of the patient. In various embodiments, the physician is to insert the assembly 940 with the trocar 838 into an incision made on the patient and to advance the assembly 940 in tissue of the patient until the trocar 838 reaches the bladder neck, and the tip of the sheath 746 is withdrawn (e.g., about 1 to 2 cm) to allow the adjustable membrane element 112 to be partially inflated (e.g., to about 1 cc) at the bladder neck while maintaining position of the tip of the sheath 746 at the target site. In one embodiment, the trocar 838 having the sharp tip is used until the sharp tip reaches the pelvic floor of the patient, and then it is replaced by the trocar 838 having the blunt tip to avoid bladder perforation when approaching the bladder neck of the patient.

In various embodiments, a surgical tool kit including the trocar 838 and the sheath 746 is provided to the physician performing implantation of the device such as implantable device 110 and 510. In one embodiment, the surgical tool kit includes the trocar 838 having the sharp tip, the trocar 838 having the blunt tip, and the sheath 746. In one embodiment, the surgical tool kit, including the trocar(s) 838 (sharp tip and/or blunt tip) and the sheath 746 is disposable, i.e., intended for single-use. Compared to reusable trocar and sheath, the disposable trocar and sheath can be more cost effective and/or safer (e.g., due to the cost and effectiveness concerns associated with the cleansing and re-sterilization).

FIG. 10 is an illustration, including a broken-out view, showing introduction of an implantable device 1010 into tissue of a male post-prostatectomy patient using the assembly 940, to provide for coaptation of a urethra, according to an embodiment of present subject matter. Examples of the implantable device 1010 include implantable devices 110, 510, 1310, 1410, 1510, and any of their embodiments as discussed in this document. In various embodiments, the physician is to insert the assembly 940 into an incision made in the perineum below the scrotum of the patient and to advance the assembly 940 in tissue of the patient until the tip of the trocar 838 reaches a target site proximal to the bladder neck of the bladder. Then, the trocar 838 is removed while maintaining position of the tip of the sheath 746 at the target site. The implantable device 1010 is advanced with the adjustable membrane element deflated through the sheath 746 to a selected position using a push wire. In one embodiment, it is helpful to ensure the push wire is fully inserted in the adjustable continence device. In one embodiment, the position of the front end of the implantable device 1010 can be confirmed by, for example, fluoroscopy, cystoscopy or palpation. In one embodiment, the tip of the implantable device 1010 is positioned adjacent the bladder neck.

In one embodiment, the sheath is pulled back about 2 centimeters such that the adjustable membrane element of the implantable device 1010 is clear of the sheath 746. This is to, in part, ensure that the balloon is not damaged during inflation. The implantable device 1010 can then be adjusted by penetrating the septum of the rear port of the implantable device 1010 with a needle of a syringe, such as a 23 gauge non-coring needle on the syringe, and the adjustable membrane element can be partially inflated with fluid, such as with approximately 1 milliliter of normal saline or isotonic contrast solution. In embodiments using x-ray visualization or ultrasound, the physician can view the adjustable membrane element assuming a spherical shape. In one embodiment, the sheath 746 is completely removed from the patient before the adjustable membrane element is inflated.

In one embodiment, after the adjustable membrane element of the implantable device 1010 is partially inflated and the sheath 746 is completely removed from the patient, a path is tunneled into the scrotum, and the rear port of the implantable device 1010 is grasped with a forceps and placed toward the top end of the scrotum. The incision can be closed, such as by suturing, over the conduit of the implantable device 1010.

FIG. 11 is an illustration, including a broken-out view, showing a pair of implantable devices 1010A and 1010B placed in the patient to provide for coaptation of the urethra, according to an embodiment of present subject matter. In various embodiments, the implantable devices 1010A and 1010B are each an instance of the implantable device 1010. The physician can apply the same procedure as discussed above to place each of the implantable devices 1010A and 1010B, with the two devices positioned contralaterally to each other with respect to the urethra of the patient.

In one embodiment, when it is possible, the physician is to confirm symmetrical positioning of the adjustable membrane elements of the implantable devices 1010A and 1010B with respect to the urethra such as by using x-ray visualization or ultrasound. The push wires can be removed from the implantable devices 1010A and 1010B after the positioning of both devices is determined to be adequate by the physician.

FIG. 12 is an illustration, including a broken-out view, showing adjustment of the coaptation of the urethra after the pair of the implantable devices 1010A and 1010B are placed in the patient, according to an embodiment of present subject matter. After their placement in the patient, the volume in the adjustable membrane elements of the implantable devices 1010A and 1010B can each be adjusted percutaneously by penetrating the skin and the septum of the rear port of each of the implantable devices 1010A and 1010B with the needle of the syringe 120. After placement at the target sites, the adjustable membrane elements are each inflated to a volume of 0.5 to 1.5 cc to observe, with x-ray or ultrasound, that they are in the correct positions to coapt the urethra. Once this is confirmed, the adjustable membrane elements are left at a volume of 0.5 to 1.5 cc each such that they remain firmly in position but not distorted, for example, by scar tissue or anatomical abnormalities. The adjustable membrane elements are left in this state for 4 to 6 weeks so that tissue encapsulation fixes their positions. Subsequently, the volume of each adjustable membrane element is adjusted upward in 0.5 to 1.0 cc every 4 to 6 weeks until continence is achieved. Over time further adjustments may be needed to increase the volume of each of the adjustable membrane elements in order to maintain continence or to decrease that volume to prevent urinary retention. In some examples, the physician optionally confirms absence of urethral or bladder injury by cystoscopic examination.

While coaptation of the urethra in a male post-prostatectomy patient is illustrated in FIGS. 10-12 as an example, the present subject matter can be applied for coaptation of the urethra of any patient for urinary continence, in various embodiments (e.g., in a female patient, or in a male patient after transurethral resection of the prostrate (TURP)). In various other embodiments, the present subject matter can be applied for coaptation of any body lumen when desired and feasible considering factors including anatomy.

FIG. 13 is an illustration of an implantable device kit 1320, including an implantable device 1310, a sheath 1346, and optionally a push wire 1324, according to an embodiment of present subject matter. The implantable device 1310, the sheath 1346, and optionally the push wire 1324 can be provided as a device kit 1320, which can also include other accessories (e.g., surgical tools for inserting the sheath 1346 into tissue, such as one or more trocars for use with the sheath, as discussed with reference to FIGS. 7-9 above). The implantable device 1310 can be used to coapt a lumen in a body, and can include an adjustable membrane element (also referred to as a balloon) 1312, an elongate conduit 1314, a rear port 1316, and a helix 1350. The adjustable membrane element 1312 is configured to coapt the lumen and includes a continuous wall having an inner surface defining a chamber. The rear port 1316 includes a conical strain relief 1325 made of a silicone-based or segmented polyurethane-based elastomer coupled to a port base 1323. The conduit 1314 has a conduit rear end 1314A coupled to the rear port 1316 within the strain relief 1325, a conduit front end 1314B coupled to the adjustable membrane element 1312, a peripheral surface connected to and sealed to the adjustable membrane element 1312 near the conduit front end 1314B, and optionally (when the push wire 1324 is used, as discussed below) a push wire lumen 1317 extending longitudinally in the conduit 1314 from a lumen inlet 1317A near the conduit rear end 1314A to a lumen front end 1317B at the conduit front end 1314B. The lumen inlet 1317A has a size allowing a portion of the push wire 1324 to enter. The push wire lumen 1317 has a diameter to accommodate at least the portion of the push wire 1324 that enters through the lumen inlet 1317A. The diameter is suitable for the push wire 1324 to move longitudinally in the push wire lumen 1317 by pushing a portion of the push wire 1324 that is outside of the conduit 1314.

The rear port 1316 is coupled to the conduit 1314 with the conduit rear end 1314A in the strain relief 1325. The port base 1323 includes a cavity (not shown in FIG. 13) in fluid communication with the chamber of the adjustable membrane element 1312 through an inflation lumen (not shown in FIG. 13) in the conduit 1314 to allow for expansion of the adjustable membrane element 1312 by injecting a fluid into the chamber and contraction of the adjustable membrane element 1312 by withdrawing the fluid from the chamber. In some embodiments, the rear port 1316 is releasably coupled to the conduit rear end 1314A. In various embodiments, the exterior surface of the port base 1323 is covered by a layer made from the same material as the strain relief. This can be done by extending the strain relief 1325 to cover a substantial portion of the port base 1323 or the entire port base 1323.

In one embodiment, the rear port 1316 is substantially identical to the rear port 116 as discussed above with reference to FIG. 2. The strain relief 1325 is formed by the tapered portion of the overmold 113. The port base 1323 is formed by the remaining portion of the overmold 113 (surrounding the port liner 111), the port liner 111, the elastic septum 118, and the cavity 116A. In other words, the strain relief 1325 includes the tapered portion of the overmold 113 of the rear port 116 as shown in FIG. 2, and the port base 1323 includes the rest of the rear port 116 as shown in FIG. 2.

In various embodiments, the implantable device 1310 is a multi-lumen (e.g., dual lumen) implantable device including the push wire lumen 1317 and the inflation lumen (not shown in FIG. 13) as separate lumens,

The helix 1350 is coupled to the conduit front end 1314B to function as a fixation mechanism that limits displacement of the implantable device 1310 in the tissue after implantation by anchoring the implantable device 1310 to the tissue. In various embodiments, the implantable device 1310 can be anchored to the tissue by turning the helix 1350 into a portion of the tissue using a rotational movement of the implantable device 1310 in a tightening direction (e.g., a clockwise direction). In some embodiments, the implantable device 1310 can be released from the tissue by disengaging the helix 1350 from the portion of the tissue using a rotational movement of the implantable device 1310 in a loosening direction (e.g., a counterclockwise direction) when, for example, the implantable device 1310 needs to be repositioned in the tissue or removed from the tissue. This may be done, for example, by introducing the push wire 1324 to rotate the implantable device 1310 or to aid such rotation. In various embodiments, the implantable device 1310 can also be released from the tissue by disengaging the helix 1350 from the portion of the tissue by pulling the implantable device 1310 (a method referred to as a “pull-out release”), with the helix 1350 being configured to avoid unacceptable level of tissue damage and/or device breakage resulting from the pulling. For example, the amount of pulling force required for pulling the implantable device 1310 from the tissue at the target site while the helix 1350 remains engaged with the tissue (referred to as the “pull-out force”) is to be small enough to prevent the implantable device 1310 from being broken and a portion left inside the patient. In one embodiment, the helix 1350 is made of a bioresorbable material to facilitate the pull-out release. After being placed in body tissue, a bioresorbable material (also referred to as biodegradable material) degrades over time into one or more non-toxic substances that can be safely absorbed by the patient's body. The bioresorbable helix 1350 can anchor the implantable device 1310 until it is stabilized by the tissue encapsulation that follows the initial placement. Examples of bioresorbable materials for the helix 1350 include bioresorbable polymers that have mechanical properties (e.g., strength and stiffness) suitable to be used to construct the helix, such as chitosan (e.g., derived from naturally occurring chitin, such as from shellfish or mushrooms), and bioresorbable metals such as alloys of manganese, magnesium, iron, and/or zinc.

The implantable device 1310 can be a combination of the helix 1350 with a suitable implantable device selected from those discussed with reference to FIGS. 1-12, including but not limited to the implantable device 110 or an implantable device including various combinations of features of the implantable devices 110, 510, and 1010.

The sheath 1346 has an elongate cylindrical sheath body 1345 and a channel 1344 extending longitudinally within the sheath body 1345. The sheath body 1345A has a sheath rear end 1345A, a sheath front end 1345B, and a slot 1348B-C extending longitudinally along at least a portion of sheath body 1345. In the illustrated embodiment, the slot 1348B-C includes a slot front portion 1348B and a slot middle portion 1348C. In other embodiments, the slot middle portion 1348C can extend to the sheath rear end 1345A or a point near the sheath rear end 1345A. The slot middle portion 1348C is sized to allow at least the adjustable membrane element 1312 (in its deflated state) and the conduit 1314 to be placed in the channel 1344 of the sheath 1346. The slot front portion 13488 is sized to prevent adjustable membrane element 1312 from exiting the channel 1344 when being advanced within the channel 1344 and to allow the sheath 1346 to be separated from the implantable device 1310 (e.g., by passing a portion of the elongate conduit 1314, which can be stretched to reduce its diameter if necessary, through the slot front portion 1348B) and then removed from the tissue after the implantable device 1310 is placed in and anchored to the tissue. This is to reduce the possibility of an edge of the slot front portion 1348B cutting into the inflation lumen in the conduit 1314. Another way (other than stretching the elongate conduit 1314) to protect the inflation lumen in the conduit 1314 from being cut by an edge of the slot front portion 1348B is to align the lumen inlet 1317A with the middle of the slot front portion 1348B when passing the portion of the elongate conduit 1314 through the slot front portion 1348B). Yet another way to protect the inflation lumen in the conduit 1314 from being cut by an edge of the slot front portion 1348B is to pass a portion of the strain relief through the slot front portion 1348B, as further discussed below with reference to FIG. 19. In various embodiments, the sheath 1346 can be a sheath 946 that is C-shaped across the diameter and modified to include the slot 1348 having a varying width, which includes a front portion and a middle portion that is wider than the front portion.

In one embodiment, the outer surface of the rear port 1316 and the adjustable membrane element 1312 are of a size (e.g., a diameter) that is smaller than an inner size (e.g., a diameter) of the channel 1344 to allow the implantable device 1310 to be moved longitudinally through the channel 1344 of the sheath 1346. In an alternative embodiment, the rear port 1316 is constructed of at least one material flexible enough to allow the size of the rear port 1316 in its relaxed state to be compressed to a size sufficiently small so that the implantable device 1310 can be moved longitudinally through the channel 1344. In one embodiment, the conduit 1314 has a stiffness sufficient to allow force applied at the conduit rear end 1314A (e.g., through the rear port 1316) to move the implantable device 1310 at least partially through the channel 1344. In this embodiment, the push wire 1324 is optional, and the implantable device kit 1320 may only include the implantable device 1310 and the sheath 1346. In one embodiment, the stiffness of the conduit 1314 is determined based on the type of material used in constructing its tubular elongate body. For example, the conduit 1314 can be made of polyurethane or silicone. The conduit 1314 made of polyurethane would be substantially stiffer than the conduit 1314 made of silicone. Alternatively, support elements can be added to the tubular elongate body of the conduit 1314. For example, a metal coil can be placed longitudinally within the tubular elongate body to increase the stiffness of the tubular elongate body. In one embodiment, the conduit 1314 can have variable stiffness along its length provided by polyurethane having various levels of stiffness.

In another embodiment, the push wire 1324 can be used to move the implantable device 1310 at least partially through the channel 1344 of the sheath. 1346. The push wire 1324 has an elongate push wire body 1326 having a push wire rear end 1326A and a push wire front end 1326B. The push wire front end 1326B can have any shape suitable for advancing the implantable device 1310 in the channel 1344 of the sheath 1346 and/or the tissue. The elongate push wire body 1326 has a diameter suitable for moving longitudinally in the push wire lumen 1317 of the conduit 1314. The longitudinal movements of the push wire 1324 includes moving the push wire 1324 along its own longitudinal axis (which is also substantially parallel to the longitudinal axis of the conduit 1314).

The push wire 1324 and the conduit 1314 can optionally be configured to allow the push wire to be used to rotate, or to aid the rotation of, the implantable device 1310. In some embodiments, as illustrated in FIG. 13, the pulse push wire front end 1326B is configured to include a driver 1327, and the conduit front end 1314B is configured to include a drive 1328 that is shaped to mate the driver 1327. For example, the driver 1327 can have a shape similar to the front end of a screw driver, and the drive 1328 can have a shape of the drive in a screwhead that corresponds to the shape of the screw driver (e.g., slot, Phillips, square, hex, or another standard or nonstandard screw drive shape). In other embodiments in which the push wire 1324 is not intended to be used for rotating the implantable device 1310, the driver 1327 and the drive 1328 are unnecessary and may not be included.

In this document, terms including “substantial”, “substantially”, “approximate”, “approximately”, or the like can refer to imperfection or inaccuracy resulting from practical factors including, but not limited to, accuracy in manual handling and errors within manufacturing tolerances. For example, the longitudinal axes of the push wire and the push wire lumen of the conduit can be “substantially parallel” when the former is partially placed in the latter because they are not perfectly parallel due to (1) errors within their manufacturing tolerances, (2) manually controlled movements of the push wire in the push wire lumen, and (3) a portion of the push wire is not in the push wire lumen, among other things. Such terms (“substantial”, “substantially”, “approximate”, “approximately”, or the like” can also refer to small deviations by design. For example, a push wire lumen can be “substantially parallel” to the longitudinal axes of the conduit while a small portion of the push wire lumen next to the inlet (on a lateral side of the conduit) deviates from being parallel to the longitudinal axes of the conduit by design. In a multi-lumen implantable device, the push wire lumen can be “substantially parallel” to the longitudinal axes of the conduit. While a major portion of this push wire lumen can be off-center in the conduit to allow space for inflation lumen, the front-end portion of the push wire lumen can deviate from being parallel to the longitudinal axes of the conduit to end at the center of the front end of the conduit.

FIG. 14 is an illustration of an implantable device kit 1420, including an implantable device 1410, the sheath 1346, and optionally the push wire 1324, according to an embodiment of present subject matter. The implantable device 1410, the sheath 1346, and optionally the push wire 1324 can be provided as a device kit 1420, which can also include other accessories (e.g., surgical tools for inserting the sheath 1346 into tissue, such as one or more trocars for use with the sheath, as discussed with reference to FIGS. 7-9 above). The implantable device 1410 can be used to coapt a lumen in a body, and can include an adjustable membrane element (also referred to as a balloon) 1412, an elongate conduit 1414, a rear port 1416, and a fixation mechanism 1350. The adjustable membrane element 1412 is configured to coapt the lumen and includes a continuous wall having an inner surface defining a chamber. The rear port 1416 includes a conical strain relief 1425 (e.g., made of silicone or biostable segmented polyurethane) coupled to a port base 1423. The conduit 1414 has a conduit rear end 1414A coupled to the rear port 1416 within the strain relief 1425, a conduit front end 1414B coupled to the adjustable membrane element 1412, a peripheral surface connected to and sealed to the adjustable membrane element 1412 near the conduit front end 1414B, and an inflation lumen 1415 extending longitudinally in the conduit 1414. The inflation lumen 1415 has a lumen rear opening 1415A at the conduit rear end 1414A, a lumen front opening 1415B in fluid communication with the chamber of the adjustable membrane element 1412 to allow for expansion of the adjustable membrane element 1412 by injecting a fluid into the chamber and contraction of the adjustable membrane element 1412 by withdrawing the fluid from the chamber, and a lumen front end 1415C to allow the push wire 1324 to advance the implantable device 1410 in the tissue and/or to operate fixation mechanism 1450. Lumen front end 1415C is a closed end that does not allow the fluid to leak out of the lumen 1415.

The rear port 1416 is coupled to the conduit 1414 with the conduit rear end 1414A in the strain relief 1425. The port base 1423 includes a cavity 1419 in fluid communication with the chamber of the adjustable membrane element 1412 though the inflation lumen 1415 to allow for expansion of the adjustable membrane element 1412 by injecting a fluid into the chamber and contraction of the adjustable membrane element 1412 by withdrawing the fluid from the chamber. The cavity 1419 is sealed by a septum 1418 that is elastic and self-sealing after being pierced through, for example by a hollow needle coupled to a syringe for injecting and withdrawing the fluid, In some embodiments, the rear port 1416 is releasably coupled to the conduit rear end 1414A. In various embodiments, the exterior surface of the port base 1423 is covered by a lining made of a material such as a silicone- or polyurethane-based copolymer. For example, the lining can include a thin layer formed by extending the strain relief 1425 to cover a substantial portion of the port base 1423 or the entire port base 1423.

In one embodiment, the outer surface of the rear port 1416 and the adjustable membrane element 1412 are of a size (e.g., a diameter) that is smaller than an inner size (e.g., a diameter) of the channel 1344 to allow the implantable device 1310 to be moved longitudinally through the channel 1344 of the sheath 1346. In an alternative embodiment, the rear port 1416 is constructed of at least one material flexible enough to allow the size of the rear port 1416 in its relaxed state to be compressed to a size sufficiently small so that the implantable device 1410 can be moved longitudinally through the channel 1344. In one embodiment, the conduit 1414 has a stiffness sufficient to allow force applied at the conduit rear end 1414A (e.g., through the rear port 1316) to move the implantable device 1410 at least partially through the channel 1344. In this embodiment, the push wire 1324 is optional, and the implantable device kit 1420 may only include the implantable device 1410 and the sheath 1346. In one embodiment, the stiffness of the conduit 1414 is determined based on the type of material used in constructing its tubular elongate body. For example, the conduit 1414 can be made of polyurethane or silicone. The conduit 1414 made of polyurethane would be substantially stiffer than the conduit 1414 made of silicone. Alternatively, support elements can be added to the tubular elongate body of the conduit 1414. For example, a metal coil can be placed longitudinally within the tubular elongate body to increase the stiffness of the tubular elongate body. In one embodiment, the conduit 1414 can have variable stiffness along its length provided by polyurethane having various levels of stiffness.

In another embodiment, the push wire 1324 can be used to move the implantable device 1310 at least partially through the channel 1344 of the sheath 1346. The implantable device 1410 can be a single-lumen implantable device with the inflation lumen 1415 also functioning as a push wire lumen. The inflation lumen 1415 can meet the requirements for the push wire lumen 1317 as discussed above, with the push wire lumen inlet being the inflation lumen rear end 1415A. The push wire 1324 can enter inflation lumen 1415 by piercing through the septum 1418.

The helix 1350 is coupled to the conduit front end 1414B to function as a fixation mechanism that limits displacement of the implantable device 1410 in the tissue after implantation by anchoring the implantable device 1410 to the tissue. In various embodiments, the implantable device 1410 can be anchored to the tissue by extending the helix 1350 into a portion of the tissue using a rotational movement of the implantable device 1410 in a tightening direction (e.g., a clockwise direction). In some embodiments, the implantable device 1410 can be released from the tissue by disengaging the helix 1350 from the portion of the tissue using a rotational movement of the implantable device 1410 in a loosening direction (e.g., a counterclockwise direction) when, for example, the implantable device 1410 needs to be repositioned in the tissue or removed from the tissue. This may be done, for example, by introducing the push wire 1324 to rotate the implantable device 1410 or to aid such rotation. In various embodiments, the implantable device 1410 can also be released from the tissue by disengaging the helix 1350 from the portion of the tissue by pulling the implantable device 1410 (i.e., the pull-out release), with the helix 1350 being configured to avoid unacceptable level of tissue damage and/or device breakage resulting from the pulling. For example, the amount of pull-out force is to be small enough to prevent the implantable device 1410 from being broken and leaving a portion inside the patient.

The push wire 1324 and the conduit 1414 can optionally be configured to allow the push wire to be used to rotate, or to aid the rotation of, the implantable device 1410. In some embodiments, as illustrated in FIG. 14, the pulse push wire front end 1326B is configured to include the driver 1327, and the conduit front end 1414B is configured to include a drive 1428 that is shaped to mate the driver 1327. For example, the driver 1327 can have a shape similar to the front end of a screw driver, and the drive 1428 can have a shape of the drive in a screwhead that corresponds to the shape of the screw driver (e.g., slot, Phillips, square, hex, or another standard or non-standard screw drive shape). In other embodiments in which the push wire 1324 is not intended to be used for rotating the implantable device 1310, the driver 1327 and the drive 1428 are unnecessary and may not be included.

The implantable device 1410 can be combination of the helix 1350 with a suitable implantable device selected from those discussed with reference to FIGS. 1-12, including but not limited to the implantable device 510 or an implantable device including various combinations of features of the implantable devices 110, 510, and 1010.

In various embodiments, the implantable device 1310 and the 1410 implantable device can have substantially similar sizes. For example, adjustable membrane element 1312 and adjustable membrane element 1412 can have substantially similar sizes, the elongate conduit 1314 and the elongate conduit 1414 can have substantially similar sizes, and the rear port 1316 and the rear port 1416 can have substantially similar sizes.

FIGS. 15-19 are illustrations of a method for placing an implantable device 1510 into tissue of a patient using the sheath 1346, according to an embodiment of present subject matter. Implantable device 1510 can be used to coapt a lumen in a body and can include an adjustable membrane element (also referred to as a balloon) 1512, an elongate conduit 1514, a rear port 1516, and a helix 1350. Examples of the implantable device 1510 includes the implantable device 1310 (with the adjustable membrane element 1512, the elongate conduit 1514, the rear port 1516 corresponding to the adjustable membrane element 1312, the elongate conduit 1314, the rear port 1316, respectively) and the implantable device 1410 (with the adjustable membrane element 1512, the elongate conduit 1514, the rear port 1516 corresponding to the adjustable membrane element 1412, the elongate conduit 1414, the rear port 1416, respectively). The embodiment as illustrated in FIGS. 15-19 is discussed by way of example, but not by way of limitation, to show how the implantable device 1510 can be placed in and anchored to the tissue. For example, while no push wire is used in the illustrated embodiment, a push wire can be used in various embodiments in which the implantable device 1510 is configured to receive a portion of the push wire for advancing the implantable device 1510 in the channel 1344 of the sheath 1346 and/or the tissue. During the performance of the method, portions of the implantable device 1510 and the sheath 1346 that has entered the patient can be seen using a medical imaging technique such as fluoroscopy or ultrasound.

FIG. 15 shows that the implantable device 1510 is partially placed in the channel 1344 of the sheath 1346 through the slot middle portion 1348C, after the sheath is placed to allow placement of the adjustable membrane element 1512 at a target site of the patient. The sheath 1346 has a sheath rear portion including the slot rear portion 1348A, a sheath front portion including the slot front end 1348B, and a sheath middle portion including the slot middle portion 13480. The slot middle portion 1348C is sized to allow the adjustable membrane element 1512 and the elongate conduit 1514 to be placed in the channel 1344 when the adjustable membrane element 1512 is substantially deflated. The slot front portion 1348B is substantially narrower than the slot middle portion 1348C to form a substantially closed portion of the channel 1344 at the sheath front portion to guide the substantially deflated adjustable membrane element 1512 into the sheath front portion. The narrower slot front portion 1348B also prevents the adjustable membrane element 1512 from exiting the channel 1344 when being advanced within the channel 1344 at the sheath front portion.

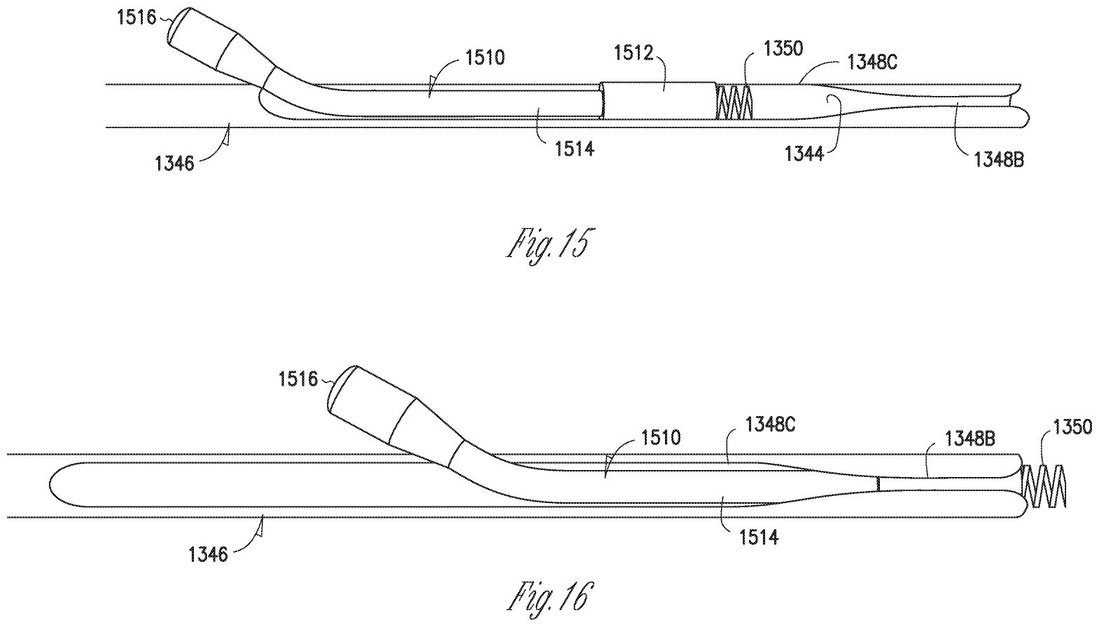

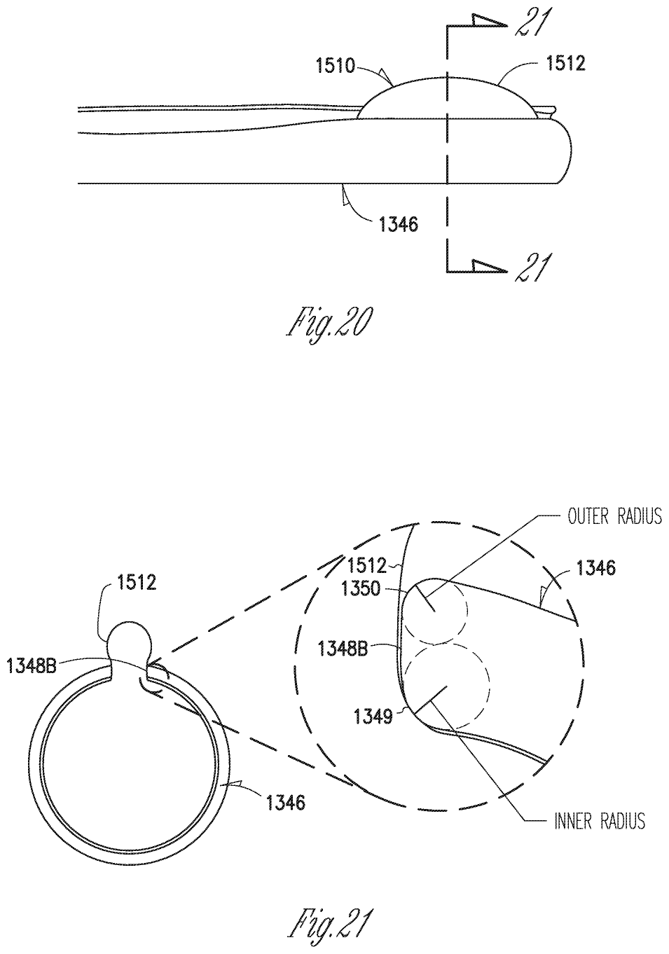

FIG. 16 shows that the adjustable membrane element 1512 of the implantable device 1510 advanced into the sheath front portion (which includes the slot front portion 1348) such that the helix 1350 extends from the sheath 1346, as can be seen using fluoroscopy or ultrasound. The adjustable membrane element 1512 is inflated to an extent such that it fills the portion of the channel 1344 in the sheath front portion and grips the inside of the sheath so that it rotates with the sheath 1346 when the sheath 1346 is rotated thus turning helix 1350 attached to conduit 114 or 514 into the tissue for fixation. This requires a relatively small volume (e.g., on the order of 0.1 cc). Over-inflation at this point may cause a portion of the adjustable membrane element 1512 to protrude from (or bulge out of) the sheath 1346 through the slot front portion 1348B, a scenario further discussed below with reference to FIGS. 20 and 21.

Due to the high sensitivity to the volume of the adjustable membrane element 1512 for resisting rotation (i.e., slipping) of the adjustable membrane element 1512 relative to the sheath with substantially limited protrusion through the slot front portion 1348B, inflation the adjustable membrane element 1512 at this point of performance of the method needs to be precisely controlled. In one embodiment, a small volume syringe, such as 1.0 cc, can be used for fine volume control in inflating the adjustable membrane element 1512. In another embodiment, the adjustable membrane element 1512 can be inflated to a specified pressure. This can be done, for example, using a pressure gauge on a syringe or a T connector coupled between the syringe and the adjustable membrane element 1512. By controlling the pressure rather than the volume, the method can be performed with a single syringe for inflating the adjustable membrane element 1512 for the rotational stability first and the coaptation of the body lumen later. In various embodiments, when desired, this volume sensitivity can be reduced with a looser fit, either by increasing the diameter of the sheath 1346 and/or decreasing the diameter of the adjustable membrane element 1512 (in its deflated state).

In various embodiments, the adjustable membrane element 1512 can be configured for desirable characteristics related to its protrusion through a slot. In various embodiments, the rotational stability (i.e., slipping resistance) of the adjustable membrane element 1512 in the sheath 1346 can be increased by incorporating gripping features to a portion of the channel 1344 in the sheath front portion (with the slot front portion 1348B) to prevent the adjustable membrane element 1512 from slipping in the sheath front portion. Examples of such features include texture and shallow longitudinal grooves or ridges on surface of the portion of the channel 1344 (i.e., the interior surface of the sheath front portion).

After the adjustable membrane element 1512 is fixed in the sheath front portion to prevent its rotation relative to the sheath 1346 with the helix 1350 extending into the tissue at the target site, the sheath 1346 is rotated in a tightening direction to turn the helix 1350 into the tissue. The number of rotations needed to provide a desirable level of fixation can be determined with testing and experience for each of different types of the tissue that the implantable device 1510 can anchor into (e.g., scar, muscle, or fat). The desirable level of fixation can provide the implantable device 1510 with sufficient fixation to prevent the adjustable membrane element 1512 from migration until encapsulation has occurred, In various embodiments, the desirable level of fixation also allowing for removal of implantable device 1510 by the pull-out release (without actively disengaging the helix 1350 from the tissue) without causing tissue damage and/or device breakage.

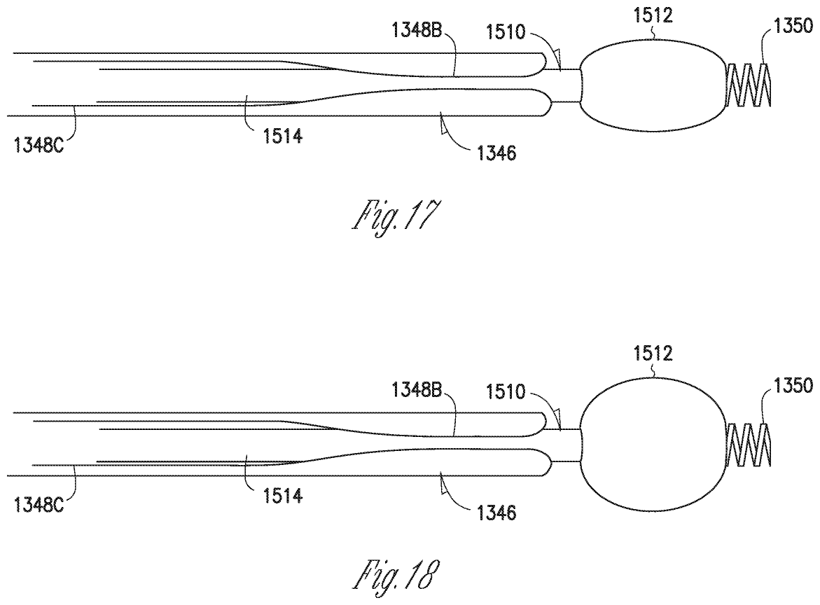

FIG. 17 shows that the sheath 1346 is withdrawn to allow inflation of the adjustable membrane element 1512 after the helix 1350 has been turned into the tissue. The adjustable membrane element 1512 is deflated to allow the withdrawal of the sheath 1346 before it is inflated in tissue.

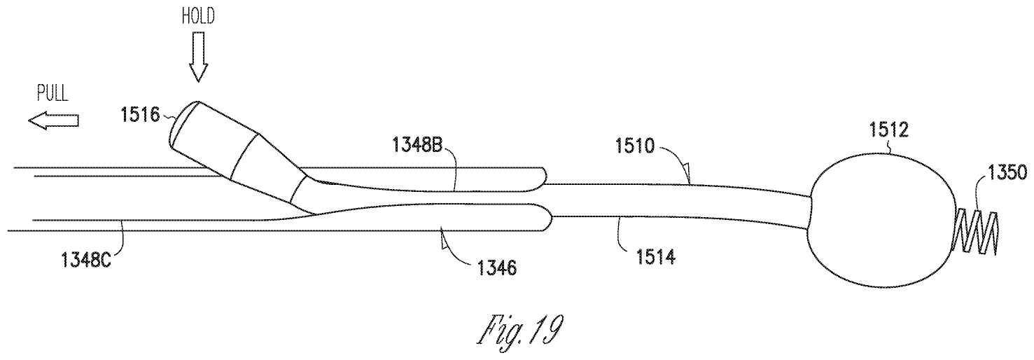

FIG. 18 shows that the adjustable membrane element 1512 is inflated (e.g., to a volume of 2-3 cc) at the tissue site. Proper positioning of the adjustable membrane element 1512 can be confirmed by observing coaptation (e.g., flattening) of the body lumen (e.g., urethra) with the fluoroscopy or endoscopy. If the position is to be adjusted, the adjustable membrane element 1512 can be deflated, returned into the sheath front portion, inflated to re-fix the adjustable membrane element 1512 in the sheath front portion to prevent it from rotating relative to the sheath 1346, and actively disengage the helix 1350 from the tissue by rotating the sheath 1346 in the loosening direction. The steps as discussed above with reference to FIGS. 16-18 can be repeated until the positioning of the adjustable membrane element 1512 is satisfactory. Alternatively, the steps as discussed above with reference to FIGS. 15-18 can be partially performed, by skipping the turning of the helix 1350, until the proper positioning of the adjustable membrane element 1512 is confirmed. Then, the adjustable membrane element 1512 is deflated, the sheath 1346 is advanced such that the sheath front portion is over the adjustable membrane element 1512, and the steps as discussed above with reference to FIGS. 16-18 can be performed to anchor the implantable device 1510 into the tissue at the target site.

FIG. 19 shows that the sheath 1346 is withdrawn until the rear port 1516 reaches the transition between the slot middle portion 1348C and the slot front portion 1348B, after the implantable device 1510 is anchored into the tissue at the target site and the proper positioning of the adjustable membrane element 1512 is confirmed. As illustrated in FIG. 19, the rear port 1516 has a conical strain relief at the transition between the rear port 1516 and the conduit 1514. In one embodiment, the transition between the slot middle portion 1348C and the slot front portion 1348B engages the conical strain relief of the port 1516 to prevent damage to the conduit 1514 while the port 1516 is pushed and/or the sheath 1346 is pulled to separate the implantable device 1510 form the sheath 1346. The strain relief can have enough elastic bulk to resist any significant damage. In another embodiment, the conduit 1514 and the sheath 1346 are configured to allow the conduit 1514 to be stretched to have a diameter smaller than the width of the slot front portion 1348B. The sheath 1346 can then be separated from the implantable device 1510 by passing the stretched conduit 1514 through the slot front portion 1348B. In another embodiment, in which the slot front portion 1348B is replaced by a slit or a very narrow slot, the slit or narrow slot can be opened using a tool (e.g., internal snap ring plyers) to allow the sheath 1346 to be separated from the implantable device 1510.

After the implantable device 1510 is placed in and anchored to the tissue at the target site, and the sheath 1346 is separated from the implantable device 1510, the implantable device may need to be removed from the patient, for example for device repositioning or replacement when the patient's condition has changes and/or when a more suitable device is available. In one embodiment, the sheath 1346 can be inserted into the patient and engaged with the implantable device 1510, for example by placing an exposed section of the conduit 1514 through the slot front portion 1348B and advancing the sheath 1346 until the adjustable membrane element 1512 (which has been deflated) is within the sheath front portion. The adjustable membrane element 1512 is then inflated to the extent allowing the helix 1350 to be actively disengaged from the tissue by rotating the sheath 1346 in the loosening direction. In another embodiment, the implantable device 1510 is removed using the pull-out release. In one embodiment, the helix 1350 is made of a bioresorbable material, as discussed above, which makes removal of the implantable device 1510 by the pull-out release (or any pulling method for removing for repositioning the implantable device 1510) easier and safer after the helix 1350 substantially degrades.

FIGS. 20-21 are illustrations of a scenario in performing the method of FIGS. 15-19 and device features related to the scenario, according to an embodiment of present subject matter. FIG. 20 shows the adjustable membrane element 1512 of the implantable device 1510 protruding from a portion of the slot of the sheath 1346 (e.g., the slot front portion 1348B). FIG. 21 is a longitudinal cross-sectional view of the adjustable membrane element 1512 from the slot front portion 1348B of the sheath 1346. As discussed above with reference to FIG. 16, over-inflation of the adjustable membrane element 1512 may cause a portion of it to protrude from the sheath 1346 through the slot front portion 1348B, thereby exposing the adjustable membrane element 1512 to the risk of damage from edges of the slot front portion 1348B. Such protrusion can be observed, for example, using fluoroscopy. On the other hand, limited protrusion of the adjustable membrane element 1512 from the slot front portion 1348B can add rotational stability (i.e., slipping resistance) of the adjustable membrane element 1512 in the sheath 1346. In this instance, extra care should be taken so that the edges of the slot be smooth and rounded. In various embodiments, as illustrated in FIG. 21, the slot front portion 1348B has two slot edges each including a round inner edge 1349 (directly coupled to the interior surface of the sheath 1346) having an inner radius and a round outer edge 1350 (directly coupled to the exterior surface of the sheath 1346) having an outer radius. The inner radius and the outer radius can be experimentally determined for preventing the adjustable membrane element 1512 from damages caused by the protrusion of the adjustable membrane element 1512 from the sheath 1346 through the slot front portion 1348B, with the inner radius being larger than the outer radius. In various embodiments, the entire slot 1348 can have such inner and outer edges. Additionally, the edges of the slot 1348, or the entire elongate cylindrical sheath body 1345, can be treated with a lubricous coating such as parylene to further prevent the adjustable membrane element 1512 from being damaged.

In various embodiments, the volume of the adjustable membrane element 1512 during each step in performing the method illustrated in FIGS. 15-19 can be empirically determined to ensure that each step can be performed as intended. Various factors determining an adequate volume for each step including, but not limited to, the torque needed to rotate the implantable device 1510 with the sheath 1346, the amount of protrusion of the adjustable membrane element 1512 from the sheath 1346 through the slot front portion 1348B that is allowed and/or desired, characteristics of the interior surface of the sheath 1346, and/or durability of the adjustable membrane element 1512.

Some non-limiting examples (Examples 1-21) of the present subject matter are provided as follows:

In Example 1, an implantable device configured to be positioned in tissue of a living body for coaptation of a body lumen of the living body is provided. The implantable device may include an adjustable membrane element, an elongate conduit, a rear port, and a helix. The adjustable membrane element may be configured to coapt the body lumen and including a continuous wall having an inner surface defining a chamber. The elongate conduit may include a conduit peripheral surface, a conduit rear end, a conduit front end, and one or more conduit lumens The conduit peripheral surface may be connected to and sealed to the adjustable membrane element at or near the conduit front end. The one or more conduit lumens may include at least an inflation lumen having a first opening at the conduit rear end, a second opening in fluid communication with the chamber, and a closed end at or near the conduit front end. The rear port may be connected to the elongate conduit at the conduit rear end and include a cavity in fluid communication with the first opening of the inflation lumen. The helix may be coupled to the conduit front end and configured to anchor the implantable device to the tissue by rotating the entire implantable device in a tightening direction.

In Example 2, the subject matter of Example 1 may optionally be configured such that the rear port includes a strain relief and a port base coupled to the strain relief and is connected to the elongate conduit with the conduit rear end in the strain relief.

In Example 3, the subject matter of any one or any combination of Examples 1 and 2 may optionally be configured such that the implantable device includes one or more elastic portions each constructed of biostable segmented polyurethane.

In Example 4, the subject matter of any one or any combination of Examples 1 to 3 may optionally be configured such that the helix is constructed of a bioresorbable material.

In Example 5, an implantable device kit for controllable coaptation of a body lumen in tissue of a target site in a living body is provided. The implantable device kit may include an implantable device and a sheath. The implantable device may include an adjustable membrane element, an elongate conduit, a rear port, and a helix. The adjustable membrane element may be configured to coapt the body lumen and include a continuous wall having an inner surface defining a chamber. The elongate conduit may include a conduit peripheral surface, a conduit rear end, a conduit front end, and one or more conduit lumens. The conduit peripheral surface may be connected to and sealed to the adjustable membrane element at or near the conduit front end. The one or more conduit lumens may include an inflation lumen having a first opening at the conduit rear end, a second opening in fluid communication with the chamber, and a closed end at or near the conduit front end. The rear port may be connected to the conduit rear end and include a cavity in fluid communication with the first opening of the inflation lumen. The helix may be coupled to the conduit front end and configured to anchor the implantable device to the tissue. The sheath may be configured to accommodate portions of the implantable device including the adjustable membrane element, to guide the implantable device to the target site, and to be used to rotate the implantable device when the portions of the implantable device is placed in the sheath with the adjustable membrane element partially inflated.

In Example 6, the subject matter of Example 5 may optionally be configured to further include a push wire and configured such that the one or more conduit lumens further include a push wire lumen having an opening on the elongate conduit to allow the push wire to enter the push wire lumen and a closed end at or near the conduit front end to allow the implantable device to be pushed forward through the sheath by applying a forwarding force to the push wire.

In Example 7, the subject matter of any one or any combination of Examples 5 and 6 may optionally be configured such that the sheath includes an elongated body and a longitudinal slot. The elongated body includes a sheath rear portion, a sheath front portion, and a sheath middle portion coupled between the sheath rear portion and the sheath front portion. The longitudinal slot includes at least a slot middle portion extending on the sheath middle portion and a slot front portion extending on the sheath front portion. The slot middle portion is configured to allow placement of the portions of the implantable device in the sheath. The slot front portion is configured to allow the implanted device to rotate with the sheath when the adjustable membrane element is placed substantially in the sheath and partially inflated and to allow the sheath to be separated from the implantable device.

In Example 8, the subject matter of Example 7 may optionally be configured such that the sheath front portion includes an interior surface including one or more gripping features.

In Example 9, the subject matter of Example 8 may optionally be configured such that the one or more gripping features include longitudinal grooves or ridges.

In Example 10, the subject matter of any one or any combination of Examples 7 to 9 may optionally be configured such that the rear port of the implantable device includes a strain relief and a port base coupled to the strain relief and is connected to the elongate conduit with the conduit rear end in the strain relief, and at least a portion the strain relief is configured for passing the slot front portion of the sheath when the sheath is separated from the implantable device.

In Example 11, the subject matter of any one or any combination of Examples 7 to 10 may optionally be configured such that the sheath includes an interior surface and an exterior surface, the longitudinal slot is formed by two slot edges each coupled between the interior surface and an exterior surface, the two slot edges each include an inner edge directly coupled to the interior surface and having an inner radius and an outer edge directly coupled to the exterior surface and having an outer radius, and the inner radius is larger than the outer radius at least for the slot front portion.

In Example 12, the subject matter of any one or any combination of Examples 5 to 11 may optionally be configured such that the implantable device includes one or more elastic portions each constructed of biostable segmented polyurethane.

In Example 13, a method for coapting a body lumen in tissue of a target site in a living body is provided. The method may include providing an implantable device. The implantable device may include an adjustable membrane element, an elongate conduit, a rear port, and a helix. The adjustable membrane element may be configured to coapt the body lumen and including a continuous wall having an inner surface defining a chamber. The elongate conduit may include a conduit peripheral surface, a conduit rear end, a conduit front end, and one or more conduit lumens The conduit peripheral surface may be connected to and sealed to the adjustable membrane element at or near the conduit front end. The one or more conduit lumens may include an inflation lumen having a first opening at the conduit rear end, a second opening in fluid communication with the chamber, and a closed end at or near the conduit front end. The rear port may be connected to the conduit rear end and including a cavity in fluid communication with the first opening of the inflation lumen. The helix may be coupled to the conduit front end. The method may further include rotating the implantable device in a tightening direction to turn the helix into the tissue upon placement of the implantable device at the target site.

In Example 14, the subject matter of Example 13 may optionally further include disengaging the helix from the tissue by pulling the implantable device.

In Example 15, the subject matter of providing the implantable device as found in any one or any combination of Examples 13 and 14 may optionally include constructing the helix using bioresorbable material.

In Example 16, the subject matter of any one or any combination of Examples 13 to 15 may optionally further include providing a sheath and placing portions of the implantable device including the adjustable membrane element in the sheath with the helix extending from a front end of the sheath, such that rotating the implantable device includes partially inflating the adjustable membrane element so that the implantable device rotates with the sheath and rotating the sheath.

In Example 17, the subject matter of providing the sheath as found in Examples 16 may optionally include providing a sheath including an elongated body and a longitudinal slot. The elongated body includes a sheath rear portion, a sheath front portion, and a sheath middle portion coupled between the sheath rear portion and the sheath front portion. The longitudinal slot includes a slot middle portion extending in the sheath middle portion and a slot front portion extending in the sheath front portion. The slot middle portion is wider than the slot front portion and sized to al low the placement of the portions of the implantable device in the sheath. The slot front portion is sized to allow the sheath to be separated from the implantable device by passing a portion of the elongate conduit through the slot front portion.

In Example 18, the subject matter of providing the sheath as found in any one or any combination of Examples 16 and 17 may optionally include providing a disposable sheath.

In Example 19, the subject matter of partially inflating the adjustable membrane element so that the implantable device rotates with the sheath as found in any one or any combination of Examples 16 to 18 may optionally include injecting a fluid into the cavity of the rear port of the implantable device and controlling a volume of the fluid being injected into the cavity to cause a portion of the adjustable membrane element of the implantable device to protrude through the slot front portion when the adjustable membrane element is placed in the sheath front portion.

In Example 20, the subject matter of partially inflating the adjustable membrane element so that the implantable device rotates with the sheath as found in any one or any combination of Examples 16 to 18 may optionally include injecting a fluid into the cavity of the rear port of the implantable device and controlling a pressure of the fluid being injected into the cavity.

In Example 21, the subject matter of rotating the implantable device in the tightening direction to turn the helix into the tissue as found in any one or any combination of Examples 13 to 20 may optionally include controlling an amount of the rotation based on a type of the tissue.

This application is intended to cover adaptations or variations of the present subject matter. It is to be understood that the above detailed description is intended to be illustrative, and not restrictive. Other embodiments will be apparent to those of skill in the art upon reading and understanding the above description. The scope of the present subject matter should be determined with reference to the appended claims, along with the full scope of legal equivalents to which such claims are entitled.

What is claimed is:

1. An implantable device configured to be positioned in tissue of a living body for coaptation of a body lumen of the living body, the implantable device comprising:
an adjustable membrane element configured to coapt the body lumen and including a continuous wall having an inner surface defining a chamber;
an elongate conduit including a conduit peripheral surface, a conduit rear end, a conduit front end, and one or more conduit lumens, the conduit peripheral surface connected to and sealed to the adjustable membrane element at or near the conduit front end, the one or more conduit lumens including at least an inflation lumen having a first opening at the conduit rear end, a second opening in fluid communication with the chamber, and a closed end at or near the conduit front end;
a rear port connected to the elongate conduit at the conduit rear end and including a cavity in fluid communication with the first opening of the inflation lumen; and
a helix coupled to the conduit front end and configured to anchor the implantable device to the tissue by rotating the entire implantable device in a tightening direction.

2. The implantable device of claim 1, wherein the rear port comprises a strain relief and a port base coupled to the strain relief and is connected to the elongate conduit with the conduit rear end in the strain relief.

3. The implantable device of claim 1, comprising one or more elastic portions each constructed of biostable segmented polyurethane.

4. The implantable device of claim 1, wherein the helix is constructed of a bioresorbable material,

5. An implantable device kit for controllable coaptation of a body lumen in tissue of a target site in a living body, comprising:
an implantable device including:
an adjustable membrane element configured to coapt the body lumen and including a continuous wall having an inner surface defining a chamber;
an elongate conduit including a conduit peripheral surface, a conduit rear end, a conduit front end, and one or more conduit lumens, the conduit peripheral surface connected to and sealed to the adjustable membrane element at or near the conduit front end, the one or more conduit lumens including an inflation lumen having a first opening at the conduit rear end, a second opening in fluid communication with the chamber, and a closed end at or near the conduit front end;
a rear port connected to the conduit rear end and including a cavity in fluid communication with the first opening of the inflation lumen; and
a helix coupled to the conduit front end and configured to anchor the implantable device to the tissue; and
a sheath configured to accommodate portions of the implantable device including the adjustable membrane element, to guide the implantable device to the target site, and to be used to rotate the implantable device when the portions of the implantable device is placed in the sheath with the adjustable membrane element partially inflated.

6. The implantable device kit of claim 5, further comprising a push wire, and wherein the one or more conduit lumens further include a push wire lumen having an opening on the elongate conduit to allow the push wire to enter the push wire lumen and a closed end at or near the conduit front end to allow the implantable device to be pushed forward through the sheath by applying a forwarding force to the push wire.

7. The implantable device kit of claim 5, wherein the sheath comprises:
an elongated body including a sheath rear portion, a sheath front portion, and a sheath middle portion coupled between the sheath rear portion and the sheath front portion; and
a longitudinal slot including at least a slot middle portion extending on the sheath middle portion and a slot front portion extending on the sheath front portion, the slot middle portion configured to allow placement of the portions of the implantable device in the sheath, the slot front portion configured to allow the implanted device to rotate with the sheath when the adjustable membrane element is placed substantially in the sheath and partially inflated and to allow the sheath to be separated from the implantable device.

8. The implantable device kit of claim 7, wherein the sheath front portion comprises an interior surface including one or more gripping features.

9. The implantable device kit of claim 8, wherein the one or more gripping features comprise longitudinal grooves or ridges.

10. The implantable device kit of claim 7, wherein the rear port of the implantable device comprises a strain relief and a port base coupled to the strain relief and is connected to the elongate conduit with the conduit rear end in the strain. relief, and at least a portion the strain relief is configured for passing the slot front portion of the sheath when the sheath is separated from the implantable device.

11. The implantable device kit of claim 7, wherein the sheath comprises an interior surface and an exterior surface, the longitudinal slot is formed by two slot edges each coupled between the interior surface and an exterior surface, the two slot edges each comprise an inner edge directly coupled to the interior surface and having an inner radius and an outer edge directly coupled to the exterior surface and having an outer radius, and the inner radius is larger than the outer radius at least for the slot front portion.

12. The implantable device kit of claim 7, wherein the implantable device comprises one or more elastic portions each constructed of hiostable segmented polyurethane.

13. A method for coapting a body lumen in tissue of a target site in a living body, the method comprising:
providing an implantable device including:
an adjustable membrane element configured to coapt the body lumen and including a continuous wall having an inner surface defining a chamber;
an elongate conduit including a conduit peripheral surface, a conduit rear end, a conduit front end, and one or more conduit lumens, the conduit peripheral surface connected to and sealed to the adjustable membrane element at or near the conduit front end, the one or more conduit lumens including an inflation lumen having a first opening at the conduit rear end, a second opening in fluid communication with the chamber, and a closed end at or near the conduit front end;
a rear port connected to the conduit rear end and including a cavity in fluid communication with the first opening of the inflation lumen; and
a helix coupled to the conduit front end; and
rotating the implantable device in a tightening direction to turn the helix into the tissue upon placement of the implantable device at the target site.

14. The method of claim 13, further comprising disengaging the helix from the tissue by pulling the implantable device.

15. The method of claim 14, wherein providing the implantable device comprises constructing the helix with bioresorbable material.

16. The method of claim 13, further comprising:
providing a sheath; and
placing portions of the implantable device including the adjustable membrane element in the sheath with the helix extending from a front end of the sheath,
wherein rotating the implantable device comprises:
partially inflating the adjustable membrane element so that the implantable device rotates with the sheath; and
rotating the sheath.

17. The method of claim 16, wherein providing the sheath comprises providing a sheath including:
an elongated body including a sheath rear portion, a sheath front portion, and a sheath middle portion coupled between the sheath rear portion and the sheath front portion; and
a longitudinal slot including a slot middle portion extending in the sheath middle portion and a slot front portion extending in the sheath front portion, the slot middle portion wider than the slot front portion and sized to allow the placement of the portions of the implantable device in the sheath, the slot front portion sized to allow the sheath to be separated from the implantable device by passing a portion of the elongate conduit through the slot front portion.

18. The method of claim 17, wherein providing the sheath comprises providing a disposable sheath.

19. The method of claim 16, wherein partially inflating the adjustable membrane element so that the implantable device rotates with the sheath comprises:
injecting a fluid into the cavity of the rear port of the implantable device; and
controlling a volume of the fluid being injected into the cavity to cause a portion of the adjustable membrane element of the implantable device to protrude through the slot front portion when the adjustable membrane element is placed in the sheath front portion.

20. The method of claim 16, wherein partially inflating the adjustable membrane element so that the implantable device rotates with the sheath comprising:
injecting a fluid into the cavity of the rear port of the implantable device; and
controlling a pressure of the fluid being injected into the cavity.

21. The method of claim 13, wherein rotating the implantable device in the tightening direction to turn the helix into the tissue comprises controlling an amount of the rotation based on a type of the tissue.
